(19) United States
(12) Patent Application Publication
(10) Pub. No.: US 2024/0225978 A1
(43) Pub. Date: Jul. 11, 2024

(54) METHODS AND COMPOSITIONS FOR PHARMACOLOGICAL TREATMENT OF BLEPHAROPTOSIS

(71) Applicant: Christine Lynn Bokman, Fort Myers, FL (US)

(72) Inventor: Christine Lynn Bokman, Fort Myers, FL (US)

(73) Assignee: Christine Lynn Bokman, Fort Myers, FL (US)

(21) Appl. No.: 17/156,554

(22) Filed: Jan. 23, 2021

(51) Int. Cl. A61K 8/49; A61Q 19/08

(52) U.S. Cl. CPC A61K 8/4946; A61Q 19/08

(57) ABSTRACT

The present invention provides methods and compositions for the treatment of blepharoptosis with or without blepharoptosis symptoms by administering an effective dose of an α adrenergic receptor agonist formulated as a composition for topical or local administration. The disclosure further provides for methods and compositions for the improvement of the cosmetic appearance of the eyes, eyelids, eyebrows, or combination thereof. The compositions comprise an α adrenergic receptor agonist selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof.

### CROSS-REFERENCE TO RELATED APPLICATIONS

This application claims the benefit of U.S. Provisional Patent Application No. 62/965,694, filed Jan. 24, 2020, the entire content of which is incorporated herein by reference.

### GOVERNMENT INTERESTS

### Not Applicable

### PARTIES TO A JOINT RESEARCH AGREEMENT

Not Applicable

### PRIOR DISCLOSURE BY INVENTOR

Not Applicable

### INCORPORATION BY REFERENCE OF MATERIAL SUBMITTED ON A COMPACT DISC

Not Applicable

### BACKGROUND OF THE INVENTION

Blepharoptosis refers to drooping of the eyelid. The normal position of the upper eyelid is 3.0-4.0 mm above the center of the pupil. The closer the upper eyelid is to the center of the pupil (less than 3.0-4.0 mm), the greater the degree of blepharoptosis. A drooping upper eyelid, in mild cases, affects cosmetic appearance. In moderate to severe cases, blepharoptosis results in visual symptoms that include limitation of the superior visual fields and decreased contrast sensitivity. In extreme cases the eyelid completely covers the pupil and blocks vision altogether.

There are many types of blepharoptosis including congenital, which is present at birth, myogenic, neurogenic, and mechanical, but by far the most prevalent type of blepharoptosis is acquired. Aging is the most common cause of acquired blepharoptosis. It is estimated that about 20 percent of the population in the United States suffers from acquired blepharoptosis, while over half of the elderly population have been shown to demonstrate some degree of blepharoptosis.

Anatomically, there are two muscles that lift the eyelid: Müller's muscle and the levator palpebrae superioris muscle. Acquired blepharoptosis results when one or both muscles are weak. Persons with blepharoptosis compensate by several mechanisms including straining the frontalis muscle of the forehead to lift the upper eyelids, adopting a chin-up head position to improve superior visual field, and limiting down-gazing activities, such as reading, since acquired blepharoptosis worsens in downgaze.

Blepharoptosis is surgically treated by tightening the muscles—either Müller's muscle, the levator palpebrae superioris, or both (Jones L T, Quickert M H, Wobig J L. The cure of ptosis by aponeurotic repair. Arch Ophthalmol 1975; 93:629-34). Blepharoptosis that is mild and therefore deemed cosmetic because it does not affect vision is not covered by insurance and can be costly. Functional blepharoptosis, or blepharoptosis that affects vision, requires several criteria be met for insurance coverage. Surgery is not always curative and may result in an undercorrection or overcorrection of blepharoptosis, requiring a second surgery. There are also potential risks associated with surgery, including but not limited to infection, bleeding, pain, partial or complete vision loss, and risks associated with anesthesia including death.

Müller's muscle contains α adrenergic receptors that lift the eyelid when activated by a adrenergic receptor agonists (Mcauliffe-Curtin D, Buckley C. “Review of a adrenoceptor function in the eye. Eye (Lond) 1989; 3:472-6). Phenylephrine is an α adrenergic agonist commonly used by ophthalmologists to dilate the pupil during examination of the retina. A secondary side effect of phenylephrine is a mild degree of eyelid elevation due to α adrenergic receptor agonism within Müller's muscle. The effect of eyelid elevation, however, lasts less than ten minutes, while pupillary dilation and disabling light sensitivity endures for up to seven hours.

The safety and efficacy of ophthalmic preparations of several α adrenergic receptor agonists have been demonstrated in their application to treat eye redness, irritation, and allergic conjunctivitis (Dockhorn R J, Duckett R G. “Comparison of Naphcon-A and its components (naphazoline and pheniramine) in a provocative model of allergic conjunctivitis.” Curr Eye Res 1994; 13(5):319-24). Several over-the-counter preparations exist and are well tolerated by patients in various concentrations of the agonists. For example, Opcon-A® (Bausch and Lomb) is used to treat eye redness and swelling. The active ingredient and a type of α adrenergic receptor agonist, naphazoline, results in vasoconstriction of ocular blood vessels, reducing eye redness and edema. Visine® Original (Johnson & Johnson) contains tetrahydrozoline, another type of α adrenergic agonist, and is also used to treat eye redness via a similar mechanism.

Other α adrenergic agonists are utilized by clinicians for the treatment of glaucoma, including brimonidine and apraclonidine, for which the safety and low side effect profile provide for increased user compliance and patient tolerance. Brimonidine, an α₂ adrenergic agonist, is used to treat glaucoma by acting on the α₂ adrenergic receptors within the anterior chamber of the eye, reducing aqueous humor production and decreasing eye pressure.

It has been discovered by the inventor that these a agonists used to treat specific eye conditions and other α adrenergic receptor agonists can successfully be utilized to treat cosmetic and functional blepharoptosis with effects that are long-lasting and minimize light sensitivity. It has also been discovered that use of α adrenergic receptor agonists formulated as a composition in a pharmaceutically acceptable carrier can be used to improve the cosmetic appearance of the eyes, eyelids, and eyebrows of the face.

### BRIEF SUMMARY OF THE INVENTION

The present invention provides methods and compositions for the pharmacological treatment of blepharoptosis by administering an effective dose of an α adrenergic receptor agonist or a pharmaceutically acceptable salt thereof. It has been surprisingly discovered that specific α adrenergic receptor agonists result in long-lasting eyelid elevation while minimizing eye irritation and light sensitivity. The inventive approach is useful in the treatment of mild to moderate blepharoptosis without the need for surgery or in providing temporary relief of blepharoptosis until surgery is obtained. Moreover, for a small amount of blepharoptosis (such as less than 1.0 mm) that does not cause visual symptoms such as superior visual field defect or decreased contrast sensitivity, the present invention is also intended for the purposes of cosmetic use comprising administering an effective dose of an α adrenergic agonists or a pharmaceutically acceptable salt thereof.

Accordingly, in one embodiment, the invention provides methods and compositions to treat blepharoptosis that affects visual field, vision, or contrast sensitivity. In another embodiment, the invention provides methods and compositions to treat cosmetic blepharoptosis that does not cause symptomatology associated with blepharoptosis. In a further embodiment, the invention provides methods and compositions to improve the cosmetic appearance of the eyes, eyelids, eyebrows, or any combination thereof.

Embodiments of the present invention include administering a composition comprised of an α adrenergic receptor agonist or salt thereof for the treatment of functional blepharoptosis, which used herein means blepharoptosis that causes visual symptoms. In certain embodiments, the α adrenergic receptor agonist may be selected from an α₁ adrenergic receptor agonist, a selective α₂ adrenergic receptor agonist, an agonist with α₂ adrenergic receptor activity, a non-selective α₁/α₂ adrenergic receptor agonist, or any combination thereof.

Further embodiments of the present invention include administering a composition that includes an α adrenergic receptor agonist or salt thereof to improve cosmetic blepharoptosis, which used herein means blepharoptosis that does not cause visual symptoms. In certain embodiments, the α adrenergic receptor agonist may be selected from an α₁ adrenergic receptor agonist, a selective α₂ adrenergic receptor agonist, an agonist with α₂ adrenergic receptor activity, a non-selective α₁/α₂ adrenergic receptor agonist, or any combination thereof. Embodiments are also directed to methods and compositions for improvement of the cosmetic appearance of the eyes, eyelids, eyebrows, or any combination thereof by administering an α adrenergic receptor agonist or salt thereof.

In other embodiments, the α adrenergic agent is administered locally or topically with a pharmaceutically acceptable carrier as a pharmaceutical composition. The administration may be in the form of a solution, dispersion, suspension, emulsion, microemulsion, ointment, gel, hydrogel, jelly, liposome, nanoparticle, lotion, cream, paste, spray, foam, microsphere, or solid implant.

In a further embodiment, the composition is administered to the eye, eyelid, or fornix topically or locally. In one embodiment, the composition is administered at least one time per day. In another embodiment, the composition is administered up to ten times per day.

The inventor of the instant invention has found the administration of the composition minimizes light sensitivity associated with pupillary dilation, a secondary effect, or in cases of dilated eye exams, the primary effect of administering compositions with α adrenergic agonists. In a further embodiment, the administration of the composition treats or improves blepharoptosis while minimizing light sensitivity.

Another aspect of the invention provides for a composition comprising an effective amount of at least one α adrenergic receptor agonist or a pharmaceutically acceptable salt thereof formulated as a pharmaceutically acceptable composition with a pharmaceutically acceptable carrier. The α adrenergic agonist may be selected from a selective α₁ adrenergic receptor agonist, a selective α₁ adrenergic receptor agonist, an agonist with α₂ adrenergic receptor activity, a non-selective α₁/α₂ adrenergic receptor agonist, or any combination thereof.

In a further embodiment, other components or excipients may be incorporated into the composition including lubricating or thickening agents, buffering agents, pH adjustors, tonicity adjustors, stabilizers, antioxidants, preservatives, sequestering agents, components included in artificial tears, or any combination thereof.

### BRIEF DESCRIPTION OF THE DRAWINGS

FIG. 1(a-d) shows external photographs of two subjects with functional blepharoptosis in both eyes before (FIG. 1a-b) and 30 minutes after drop administration of an ophthalmic solution containing naphazoline 0.1% to both eyes (FIG. 1c-d).

FIG. 2(a-d) shows external photographs of two subjects with functional blepharoptosis in both eyes before (FIG. 2a-b) and 90 minutes after drop administration of an ophthalmic solution containing naphazoline 0.1% to both eyes (FIG. 2c-d).

FIG. 3(a-d) presents superior visual field results in four subjects with functional blepharoptosis before and 60 minutes after drop administration of an ophthalmic solution containing naphazoline 0.1% to both eyes. The solid square represents a tested visual field point that was not seen by a subject, while a dot represents a point that was seen by a subject.

FIG. 4(a-b) shows external photographs of a subject with functional blepharoptosis in the subject's left eye before (FIGS. 4a) and 60 minutes after administration of brimonidine 0.2% to the left eye (FIG. 4b).

FIG. 5(a-d) shows external photographs of a subject with cosmetic blepharoptosis of the subject's right eye before and after administration of naphazoline 0.1% ophthalmic solution to the right eye at time points 0 minutes (FIG. 5a), 30 minutes (FIG. 5b), 120 minutes (FIGS. 5c), and 3 hours (FIG. 5d) after drop administration.

FIG. 6(a-b) shows external photographs of a subject with cosmetic blepharoptosis of both eyes before (FIG. 6a) and after administration of naphazoline 0.1% ophthalmic solution to both eyes (FIG. 6b)

### DETAILED DESCRIPTION OF INVENTION

Disclosed herein are the compositions and methods for treating functional and cosmetic blepharoptosis comprising the administering of an α adrenergic receptor agonist or salt thereof.

For the purpose of describing the present invention and its various embodiments, definitions are presented below. In no way is the terminology meant to limit the scope of the invention. Scientific terms and technical vocabulary used herein have the same meanings as understood by those skilled in the art.

As used herein, “eye” means the eyeball including the intraocular contents of the anterior and posterior segments of the eye as well as the outer layers that contain these segments including the sclera, Tenon's capsule, bulbar conjunctiva, and cornea.

The “eyelid,” as used herein, means the most anterior layer that is the eyelid skin, the most posterior layer that is the conjunctiva, and all the structures and tissue in between the most anterior and posterior layers. The conjunctiva of the eyelid includes the palpebral and tarsal conjunctiva.

As used herein, “fornix” or “fornices” mean the sulcus on the internal part of the eyelid lined by palpebral conjunctiva, the anterior border of which joins the bulbar conjunctiva of the eyeball. It is also the junction between the eyelid and eyeball.

As used herein, “administering” or “administration” means delivery of the α adrenergic receptor agonist to an individual. As used herein, administering can include topical administration to the eye, conjunctiva, fornices, or eyelid skin; transdermal or dermal administration by various means such as an injection; transconjunctival administration; or intraocular administration by various means such as an ocular injection or implant.

As used herein, “topical” or “topically” or “topical administration” mean the delivery of the α adrenergic agonist to an individual via direct application to the eye, conjunctiva, fornices, eyelid skin, or any combination thereof.

As used herein, “local administration” or “locally” mean the delivery of the α adrenergic receptor agonist to an individual via means other than topical administration, such as but not limited to: injection of an implant into the eye or eyelid; administration of nanoparticles, liposomes, or microliposomes to the eye or eyelid; transdermal administration; parenteral administration; or a combination of methods that result in local administration of the composition by other known techniques.

As used herein, “blepharoptosis” means an eyelid in a position that is or appears abnormal and is not higher than normal. The prefix “blepharo-” comes from the word, “blepharon,” which means “eyelid” in Ancient Greek. The root word, “ptosis,” means literally “falling” or “a fall,” in Greek. “Ptosis” as it applies to medicine is nonspecific and may refer to the falling of any anatomical body part. For example, breast ptosis, abdominal ptosis, eyebrow ptosis all refer to falling or drooping of the anatomical parts to which they refer. The present invention applies to eyelid ptosis for which the word blepharoptosis is used throughout the specification to provide specificity.

Blepharoptosis may or may not result in symptoms. Blepharoptosis symptoms include but are not limited to decreased vision, loss of vision, visual field defect, limitation of vision on upgaze, limitation of vision on downgaze, increased tearing, increased astigmatism, and decreased contrast sensitivity. Blepharoptosis that results in symptoms may be referred to as “functional blepharoptosis.” Blepharoptosis may include an eyelid in a position that results in an undesirable cosmetic appearance in the absence of blepharoptosis symptoms, which may also more specifically be referred to as “cosmetic blepharoptosis.” In one embodiment, the present invention is used to treat or improve cosmetic blepharoptosis. Functional blepharoptosis and cosmetic blepharoptosis may be referred to collectively as “blepharoptosis.” Blepharoptosis may also refer to asymmetry between the two eyelids of a subject, such that one eyelid appears lower or higher than the other in the absence of blepharoptosis symptoms.

As used herein, “treat” or “treating” or “treatment” means that the present invention achieves a biologically desirable response either permanently or temporarily. A biologically desirable response includes but is not limited to (1) slowing down or halting the progression, aggravation or deterioration of the disease or condition; (2) ameliorating the symptoms of the disease or condition; or (3) curing the disease or condition.

As used herein, “improves” or “improvement” means the present invention achieves a desired or beneficial result including but not limited to a desired therapeutic result or a desired cosmetic result or cosmetic appearance. As used herein, “cosmetic appearance” means the outward aesthetic appearance of the eyes, face, and/or body. In one embodiment, the present invention improves blepharoptosis, which means that the eyelid position is elevated such that the individual can see more of the superior visual field, a desired therapeutic benefit associated with improvement in blepharoptosis. In another embodiment, the present invention is used to improve cosmetic blepharoptosis of an individual who desires said improvement, which means that the cosmetic improvement of specifically the eyelids desired by the individual is fully or partially obtained. In another embodiment, the present invention is used to improve the cosmetic appearance of an individual who desires said improvement, which means that the outward aesthetic appearance of the individual desired by the individual is fully or partially obtained.

As used herein, “patient” or “subject” or “individual” are interchangeable and mean a living mammal, preferably human.

As used herein, “pharmaceutical composition” or “composition” or “formulation” are used interchangeably and mean a combination of at least one active ingredient or a pharmaceutically acceptable salt thereof and at least one pharmaceutically acceptable carrier or excipient to render the composition administrable and facilitate achievement of the desired effect. In one embodiment, a composition may optionally include a buffering agent and a preservative to facilitate administration to the eye and delivery of the active ingredient, preferably an α adrenergic receptor agonist.

As used herein, “pharmaceutically acceptable carrier” or “carrier” or “excipient” means a carrier that is conventionally used in the art to improve storage, administration, and/or effectiveness of the agonist. The carrier, for example only, may include an aqueous solution, suspension, dispersion, emulsion, ointment, or gel. The pharmaceutically acceptable carrier for topical administration to the eye or fornix means that it is ophthalmically acceptable to facilitate safe and effective administration to the eye or fornix.

As used herein, “pharmaceutically acceptable salt” or “ophthalmologically acceptable salt” or “salt” means the salt of the agonist(s) that imparts the same or similar biologic activity as the agonist(s).

As used herein, a “therapeutically effective amount” or “effective amount” are used interchangeably and mean any amount of a compound, agonist, agent, or composition for a purpose to produce a desired effect. For example, in certain embodiments of the present invention, the purpose(s) may be: to ameliorate the symptoms associated with blepharoptosis, to improve the physical height or position of an eyelid, to improve the aesthetic appearance of the eyes, eyelids, or eyebrows, to slow the progression of the symptoms of blepharoptosis, and/or to cure blepharoptosis. A therapeutically effective amount may differ based on the condition or disease being treated, the desired result, the compound or agent being administered, and the route of administration. An effective amount may also differ based on the individual. Determination of the effective amount will become apparent based on these and other factors and is contemplated by the disclosure herein.

As used herein, “α adrenergic receptor agonist” or “α adrenergic agonist” are used interchangeably and mean an α adrenergic agonist, a prodrug, or pharmaceutically acceptable salt thereof and can be selected from naphazoline, tetrahydrozline, phenylephrine, xylometazoline, metoxamine, metaraminol, midodrine, desglymidodrine, cirazoline, amidephrine, apraclonidine, brimonidine, clonidine, guanfacine, guanabenz, xylazine, medetomidine, dexmedetomidine, α-methyldopa, phenylpropanolamine, propylhexedrine, amphetamine, dextroamphetamine, ephedrine, epinine, ethylnorepinephrine, levarterenol, lofexidine, methamphetamine, α-methylnorepinephrine, methylphenidate, mivazerol, moxonidine, norepinephrine, norphenylephrine, pemoline, tizanidine, epinephrine, norepinephrine, isoproterenol, dipivefrin, pseudoephedrine, and mephentermine. Selective α₁ adrenergic receptor agonists can be selected from naphazoline, tetrahydrozoline, phenylephrine, xylometazoline, methoxamine, metaraminol, midodrine, desglymidodrine, cirazoline, and amidephrine. Selective α₂ adrenergic receptor agonists can be selected from apraclonidine, brimonidine, clonidine, guanfacine, guanabenz, xylazine, medetomidine, dexmedetomidine, and α-methyldopa. Agonists with α₂ adrenergic receptor activity can be selected from phenylpropanolamine, propylhexedrine, amphetamine, dextroamphetamine, ephedrine, epinine, ethylnorepinephrine, levarterenol, lofexidine, methamphetamine, α-methylnorepinephrine, methylphenidate, mivazerol, moxonidine, norepinephrine, norphenylephrine, pemoline, and tizanidine. Non-selective α₁/α₂ adrenergic receptor agonists can be selected from epinephrine, norepinephrine, isoproterenol, dipivefrin, pseudoephedrine, and mephentermine.

Compounds described herein, including α adrenergic receptor agonists described herein, may exist as enantiomers and diastereomers. The present invention includes all such stereoisomeric forms, salts thereof, and mixtures thereof. Stereoisomers of a racemic mixture may be obtained by methods that may include but are not limited to fractional crystallization and chromatography. Stereoisomers may also be obtained by stereospecific synthesis, the methodologies of which are well known to those skilled in the art. The α adrenergic agonists may also exist as tautomers, which are also within the scope of the present invention.

According to the present invention, an α adrenergic agonist is useful in both treating functional blepharoptosis by improving symptoms thereof and improving cosmetic blepharoptosis. It has been discovered that α adrenergic agonists when topically administered to the eye, for example, in the form of a drop, raise the eyelid position while minimizing light sensitivity. When an α adrenergic agonist is topically administered to the eye in subjects with symptoms of blepharoptosis, there is an improvement in superior visual field and contrast sensitivity. In subjects without symptoms of blepharoptosis, administration of an α adrenergic agonist to the eye improves cosmetic blepharoptosis while minimizing light sensitivity. In subjects without blepharoptosis, administration of an α adrenergic agonist improves the cosmetic appearance of the eyes, eyelids, eyebrows, or any combination thereof, with minimal effects on light sensitivity.

Disclosed herein are the methods and compositions for blepharoptosis treatment comprising administering an α adrenergic agonist or salt thereof formulated in a composition. Further disclosed herein are the methods and compositions for improvement in the cosmetic appearance of the eyes, eyelids, eyebrows, or any combination thereof comprising administering an α adrenergic agonist or salt thereof formulated in a composition.

Embodiments of the present invention provide for methods and compositions to treat blepharoptosis in an individual in need thereof comprising administering a composition containing a therapeutically effective amount of an α adrenergic agonist, or pharmaceutically acceptable salt thereof, selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof. The α adrenergic agonist may be selected from naphazoline, tetrahydrozline, phenylephrine, xylometazoline, metoxamine, metaraminol, midodrine, desglymidodrine, cirazoline, amidephrine, apraclonidine, brimonidine, clonidine, guanfacine, guanabenz, xylazine, medetomidine, dexmedetomidine, α-methyldopa, phenylpropanolamine, propylhexedrine, amphetamine, dextroamphetamine, ephedrine, epinine, ethylnorepinephrine, levarterenol, lofexidine, methamphetamine, «-methylnorepinephrine, methylphenidate, mivazerol, moxonidine, norepinephrine, norphenylephrine, pemoline, tizanidine, epinephrine, norepinephrine, isoproterenol, dipivefrin, pseudoephedrine, mephentermine, salts of any one thereof, or any combination thereof. In one embodiment, the selective α1 adrenergic agonist may be selected from naphazoline, tetrahydrozline, phenylephrine, amidephrine, cirazoline, desglymidodrine, metaraminol, metoxamine, midodrine, xylometazoline, salts of any one thereof, or any combination thereof. In a preferred embodiment, the selective α1 adrenergic agonist is naphazoline, tetrahydrozline, phenylephrine, salts of any one thereof, or any combination thereof. In another embodiment, the selective α₂ adrenergic agonist is selected from α-methyldopa, apraclonidine, brimonidine, clonidine, dexmedetomidine, guanabenz, guanfacine, medetomidine, xylazine, salts of any one thereof, or any combination thereof. In a preferred embodiment, the selective α₂ adrenergic agonist is brimonidine or salt thereof. In another embodiment, the agonist with α₂ adrenergic receptor activity is selected from α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, salts of any one thereof, or any combination thereof. In yet another embodiment, the non-selective α₁/α₂ adrenergic receptor agonist is selected from dipivefrin, epinephrine, isoproterenol, mephentermine, norepinephrine, pseudoephedrine, salts of any one thereof, or any combination thereof.

Embodiments of the present invention provide for methods and compositions to improve the cosmetic appearance of the eyes, eyelids, and eyebrows in an individual in need thereof by administering a composition containing a therapeutically effective amount of an α adrenergic agonist, or pharmaceutically acceptable salt thereof, selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof. The α adrenergic agonist may be selected from naphazoline, tetrahydrozline, phenylephrine, xylometazoline, metoxamine, metaraminol, midodrine, desglymidodrine, cirazoline, amidephrine, apraclonidine, brimonidine, clonidine, guanfacine, guanabenz, xylazine, medetomidine, dexmedetomidine, α-methyldopa, phenylpropanolamine, propylhexedrine, amphetamine, dextroamphetamine, ephedrine, epinine, ethylnorepinephrine, levarterenol, lofexidine, methamphetamine, α-methylnorepinephrine, methylphenidate, mivazerol, moxonidine, norepinephrine, norphenylephrine, pemoline, tizanidine, epinephrine, norepinephrine, isoproterenol, dipivefrin, pseudoephedrine, mephentermine, salts of any one thereof, or any combination thereof. In one embodiment, the selective α₁ adrenergic agonist may be selected from naphazoline, tetrahydrozline, phenylephrine, amidephrine, cirazoline, desglymidodrine, metaraminol, metoxamine, midodrine, xylometazoline, salts of any one thereof, or any combination thereof. In a preferred embodiment, the selective α₁ adrenergic agonist is naphazoline, tetrahydrozline, phenylephrine, salts of any one thereof, or any combination thereof. In another embodiment, the selective α₂ adrenergic agonist is selected from α-methyldopa, apraclonidine, brimonidine, clonidine, dexmedetomidine, guanabenz, guanfacine, medetomidine, xylazine, salts of any one thereof, or any combination thereof. In a preferred embodiment, the selective α₂ adrenergic agonist is brimonidine or salt thereof. In another embodiment, the agonist with α₂ adrenergic receptor activity is selected from α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, salts of any one thereof, or any combination thereof. In yet another embodiment, the non-selective α₁/α₂ adrenergic receptor agonist is selected from dipivefrin, epinephrine, isoproterenol, mephentermine, norepinephrine, pseudoephedrine, salts of any one thereof, or any combination thereof.

Preferably, the administration to treat blepharoptosis comprises at least one selective α₁ adrenergic agonist, selective α₂ adrenergic agonist, an agonist with α₂ adrenergic receptor activity, or non-selective α₁/α₂ adrenergic receptor agonist formulated with a pharmaceutically acceptable carrier as a pharmaceutical composition. In another embodiment, at least one α adrenergic agonist is formulated with an ophthalmologically acceptable carrier as a pharmaceutical composition. In yet another embodiment, at least one α adrenergic agonist is formulated with an acceptable carrier as a cosmetic or dermatological composition. The administration to improve the cosmetic appearance of the eyes, eyelids, eyebrows, or any combination thereof comprises at least one selective α₁ adrenergic agonist, selective α₂ adrenergic agonist, an agonist with α₂ adrenergic receptor activity, or non-selective α₁/α₂ adrenergic receptor agonist formulated with a pharmaceutically acceptable carrier as a pharmaceutical composition. In another embodiment, at least one α adrenergic agonist is formulated with an ophthalmologically acceptable carrier as a pharmaceutical composition. In yet another embodiment, at least one α adrenergic agonist is formulated with an acceptable carrier as a cosmetic or dermatological composition

In several embodiments, the composition may be administered topically or locally in the form of a solution, dispersion, suspension, emulsion, microemulsion, ointment, gel, hydrogel, jelly, liposome, nanoparticle, lotion, cream, paste, spray, foam, microsphere, or solid implant.

In one embodiment of the present invention, the pharmaceutical composition may be administered topically to the eye, eyelids, or fornices. In a further embodiment, the composition is administered topically to the bulbar conjunctiva of the eye. In a preferred embodiment, the composition is administered topically to the superior fornix. In another embodiment, the composition is administered topically to the eyelid skin.

In a further embodiment, the composition is administered topically as a solution, dispersion, suspension, emulsion, or microemulsion in the form of a drop. In one embodiment, the drop is administered at least once per application session. In one embodiment, the drop is administered one, two, three, or up to four times during an application. In another embodiment, the composition is administered topically as a gel, ointment, hydrogel, jelly, lotion, cream, paste, or foam.

In one embodiment, the composition may be administered locally to the eye or eyelids, such as injection into the eye, eyelid skin, or deeper eyelid tissue; injection in the form of a microsphere or implant into the eye, eyelid skin, or deeper eyelid tissue; local administration of nanoparticles, liposomes, or microliposomes to the eye, eyelid, or combination thereof; or a combination of methods that result in local administration of the composition by other known techniques.

In one embodiment, the composition is administered at least once daily. In another embodiment, the composition is administered twice daily. In various embodiments, the composition is administered three, four, five, six, seven, eight, nine, or up to ten times per day. In a further embodiment, the composition is administered as a single dose, for example, as an implant, or a plurality of doses over a period of time. Administration may be one or multiple times daily, weekly, monthly, or yearly.

Modes of administration and dose regimen may be adjusted by a clinician to achieve therapeutic effectiveness to produce the desired biological response. The desired biological response may include but is not limited to improvement in blepharoptosis symptoms, for example, improvement in superior visual field; improvement in eyelid position as measured objectively by a clinician; improvement of cosmetic blepharoptosis, for example, increased eyelid height in a subject without blepharoptosis symptoms; improvement in the cosmetic appearance of the eye, eyelids, and/or eyebrows; and improvement in symmetry between eyelids. For example, as disclosed in Example 1 below, it was discovered that one drop of an ophthalmic composition containing naphazoline 0.1% administered twice daily resulted in eyelid elevation, improvement of a superior visual field defect, increased contrast sensitivity, and improved cosmetic appearance of the eyes, eyelids, and eyebrows. Elevation of the eyelids resulted in relaxation of the eyebrows, improving the cosmetic appearance of and symmetry between the eyebrows. The modifications of and variations in administration and dosing will become apparent to those skilled in the art and are contemplated herein.

Another embodiment of the present invention provides for methods and compositions for treating or improving blepharoptosis in an individual in need thereof comprising administering a therapeutically effective amount of an α adrenergic agonist or pharmaceutically acceptable salt thereof formulated as a composition that minimizes light sensitivity. Certain α adrenergic agonists cause pupillary dilation, producing symptoms of light sensitivity. Part of the method provided comprises an administering of a composition that minimizes pupillary dilation, thereby minimizing light sensitivity. In another embodiment, the invention provides for methods and compositions for improving the cosmetic appearance in an individual in need thereof without symptoms of blepharoptosis comprising administering a therapeutically effective amount of an α adrenergic agonist or pharmaceutically acceptable salt thereof formulated as a composition that minimizes light sensitivity.

A further aspect of the invention is a composition for treating or improving blepharoptosis in an individual in need thereof comprising an effective amount of at least one α adrenergic receptor agonist or a pharmaceutically acceptable salt thereof formulated as a pharmaceutically acceptable composition in a pharmaceutically acceptable carrier. In a further embodiment, the pharmaceutically acceptable carrier is an ophthalmologically acceptable carrier. In another embodiment, the composition comprising at least one α adrenergic receptor agonist or a pharmaceutically acceptable salt thereof is formulated as a pharmaceutical, ophthalmic, cosmetic, or dermatological composition for improving the cosmetic appearance of the eyes, eyelids, eyebrows, or any combination thereof.

In embodiments of the present invention, the α adrenergic agonist may be selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof. The α adrenergic agonist may be selected from naphazoline, tetrahydrozline, phenylephrine, xylometazoline, metoxamine, metaraminol, midodrine, desglymidodrine, cirazoline, amidephrine, apraclonidine, brimonidine, clonidine, guanfacine, guanabenz, xylazine, medetomidine, dexmedetomidine, α-methyldopa, phenylpropanolamine, propylhexedrine, amphetamine, dextroamphetamine, ephedrine, epinine, ethylnorepinephrine, levarterenol, lofexidine, methamphetamine, α-methylnorepinephrine, methylphenidate, mivazerol, moxonidine, norepinephrine, norphenylephrine, pemoline, tizanidine, epinephrine, norepinephrine, isoproterenol, dipivefrin, pseudoephedrine, mephentermine, salts of any one thereof, or any combination thereof.

In one embodiment, the selective α₁ adrenergic agonist may be selected from naphazoline, tetrahydrozline, phenylephrine, amidephrine, cirazoline, desglymidodrine, metaraminol, metoxamine, midodrine, xylometazoline, salts of any one thereof, or any combination thereof. In a preferred embodiment, the selective α₁ adrenergic agonist is naphazoline, tetrahydrozline, phenylephrine, salts of any one thereof, or any combination thereof. In another embodiment, the selective α₂ adrenergic agonist is selected from α-methyldopa, apraclonidine, brimonidine, clonidine, dexmedetomidine, guanabenz, guanfacine, medetomidine, xylazine, salts of any one thereof, or any combination thereof. In a preferred embodiment, the selective α₂ adrenergic agonist is brimonidine or salt thereof. In another embodiment, the agonist with α₂ adrenergic receptor activity is selected from α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, salts of any one thereof, or any combination thereof. In yet another embodiment, the non-selective α₁/α₂ adrenergic receptor agonist is selected from dipivefrin, epinephrine, isoproterenol, mephentermine, norepinephrine, pseudoephedrine, salts of any one thereof, or any combination thereof.

In one embodiment, the composition contains at least 0.01% w/v of at least one α adrenergic agonist. In various embodiments, the composition may contain 0.01%, 0.02%, 0.03%, 0.04%, 0.05%, 0.06%, 0.07%, 0.08%, 0.09%, or 0.1% w/v, or any concentration between these values, of at least one α adrenergic agonist. In various embodiments, the composition may contain 0.1%, 0.2%, 0.3%, 0.4%, 0.5%, 0.6%, 0.7%, 0.8%, 0.9%, or 1.0% w/v, or any concentration between these values, of at least one α adrenergic agonist.

In another embodiment, the composition contains a therapeutically effective amount of at least one α adrenergic agonist. As previously stated, a therapeutically effective amount means the amount that produces the desired biological response. The therapeutically effective amount can be easily determined by those skilled in the art and may be determined based on factors including but not limited to the type of α adrenergic agonist in the composition, the mode of administration, the dose regimen, and the characteristics of the subject being treated.

Embodiments of the present invention provide for pharmaceutical formulations for topical or local administration. The composition may be pharmaceutically formulated as a solution, dispersion, suspension, emulsion, microemulsions, ointment, gel, hydrogel, jelly, liposome, nanoparticle, lotion, cream, paste, spray, foam, microsphere, or solid implant. In one preferred embodiment, the composition is formulated as a lipophilic solution for topical application to the eye. In another preferred embodiment, the composition is formulated as an ointment for topical application to the eye. In yet another preferred embodiment, the composition is formulated as a cream for topical application to the eyelid. Formulations that facilitate various modes of administration including ophthalmically, transdermally, subcutaneously, and intraocularly are also within the scope of the present invention.

In certain preferred embodiments, the composition is a solution comprising an α adrenergic agonist and an excipient suitable for preparation of a suspension, for example, purified water or another ophthalmologically acceptable carrier including but not limited to alcohols, oils, glycerol, and the like. In a further embodiment, other components or excipients may be incorporated in the composition including lubricating or thickening agents, buffering agents, pH adjustors, tonicity adjustors, stabilizers, antioxidants, preservatives, sequestering agents, components included in artificial tears, and any combination thereof.

In other embodiments, the composition is formulated using an oil excipient, which may include, but not limited to, mineral oils, vegetable oils, or any combination thereof. In further embodiments, the composition is formulated as an oil-in-water or water-in-oil emulsion. Emulsifying agents may include gum acacia, gum tragacanth, soy bean, lecithin, or other naturally-occurring phosphatides.

In other embodiments, the composition comprising an α adrenergic agonist and suitable excipient is formulated for administration in the form of an ointment, gel, hydrogel, jelly, liposome, nanoparticle, lotion, cream, paste, spray, foam, microsphere, or solid implant. The present composition may be prepared using conventional dosage forms, preparations, and techniques that are well known to those skilled in the art. Examples of such known conventions can be found in publications, such as, Ansel's Pharmaceutical Dosage Forms and Drug Delivery Systems, 11^{th} Edition, Wolters Kluwer, Philadelphia (2018), which is expressly incorporated herein by reference.

In one embodiment, the composition may further comprise a demulcent selected from but not limited to hydroxyethylcellulose, hydroxyproplymethylcellulose, methylcellulose, dextran, gelatin, glycerin, gelatin, glycerin, polyethylene glycol, polyvinyl alcohol, povidone, N-vinyl pyrrolidone, or any combination thereof. Advantages of a demulcent include decreased evaporation of the composition from the surface of the eye, increased lubrication, and improved compliance with ease of use. In one embodiment, the composition comprises a demulcent as described in U.S. Pat. No. 4,421,748, the entirety of which is incorporated by reference.

In another embodiment of the present invention, the composition further comprises a buffering agent to adjust the acidity or alkalinity of the composition. In one embodiment, the pH of the present composition is adjusted to within a range of 5.0 to 8.0. In a preferred embodiment, the pH of the composition is about 5.8 to 7.8. Buffering agents used to adjust the pH may be selected from but not limited to boric acid, sodium borate, citric acid, potassium citrate, potassium bicarbonate, sodium bicarbonate, sodium hydroxide, hydrochloric acid zinc sulfate, various phosphate buffers, or any combination thereof. In a preferred embodiment, the buffer is borate or boric acid. The amount of buffering agent is that amount necessary to achieve a pH within the desired range, from about. 5.8 to 7.8.

In a further embodiment, the composition may optionally include anti-free radical agents (antioxidants) such as vitamin A (retinol), vitamin C (ascorbic acid), vitamin E (a tocopherol), vitamin K, derivatives of the aforementioned vitamins, sodium bisulfite, N-acetylcysteine salts, or other acceptable antioxidants well known to the pharmaceutical art.

In yet a further embodiment, the composition further comprises a preservative in an amount effective to preserve the composition. Such composition may be packaged in a multi-use container in amounts of about 5 mL to about 15 mL per container. The advantage of the composition containing preservatives is enhanced convenience of use by opening the container and administering the composition multiple times per container. Agents used to preserve a composition are intended to provide antimicrobial activity. Once a multi-use container is opened, it is no longer sterile, and being used multiple times, increases the risk for contamination. Preservation of a composition to decrease microbial activity is well known to those skilled in the art. Variations to preserving the composition are within the scope of the present invention. Examples of agents that may be used to preserve the composition include but are not limited to a quaternary ammonium preservative agent such as benzalkonium chloride or benzoxonium chloride, SofZia®, Purite®, benzyl alcohol, phenyl ethanol, hypochlorites, chlorates, perchlorates, sodium chlorite or other chlorite components, stabilized chlorine dioxide, alkyl mercurty salts, metal chlorites, such as alkali metal and alkaline earth metal chloride, sorbic acid, chlorobutanol, disodium edtate, thimerosal, parabens such as methyl or propyl paraben, and phenylmercuric salts.

In another embodiment, the composition is preservative free. Preferably, the preservative free composition is packaged in single-use containers in amounts of about 0.2 mL to 4 mL per container. Once opened, the preservative free composition is used, for example, comprising administration to the affected eye(s) within no longer than a 24-hour period of opening, and then discarded after use. The advantage of the preservative free composition is avoidance of the various side effects associated with preservatives that individuals may experience.

In one embodiment, the composition further contains a chelating or sequestering agent such as ethylenediaminetetraacetic acid (EDTA), the advantage of which comprises binding of mineral and metals. In another embodiment, the composition is free of EDTA. Other chelating agents include but are not limited to alkali metal hexametaphosphate, citric acid, sodium citrate, and mixtures thereof.

The composition may also include an enhancer of conjunctival, forniceal, corneal, ocular, or cutaneous penetration to facilitate delivery of the α adrenergic agonist to the target tissue. An enhancer may improve the absorption, increase the therapeutic effect, enhance the cosmetic result, and/or decrease systemic side effects of the agonist.

In some embodiments, formulations comprising the present invention may also include other components well known to the art of pharmaceutical science that include but are not limited to pharmaceutically acceptable diluents, fillers, binders, surfactants, hydrophobic carriers, hydrophilic carriers, emulsifiers, solubilizers, humectants, moisturizers, or any combination thereof. Solubilizers may include but are not limited to polysorbate 20, polysorbate 80, fatty acid glycerol polyethylene glycol esters, polyethylene glycols, a cyclodextrin, or any combination thereof.

In certain embodiments, the composition may optionally comprise another active agent that enhances or augments the improvement in blepharoptosis when administered to an individual in need thereof. In a further embodiment, the composition may comprise another active agent that adds an additional beneficial therapeutic effect when administered.

In another embodiment, the composition may further comprise components commonly included in artificial tears. These include but are not limited to polyvinyl alcohol, polyols, dextrans, cellulose derivatives, carbomers, and other components described in U.S. Pat. Nos. 5,209,927 and 6,348,508, which are incorporated by reference for such disclosure in their entirety.

The effective concentrations for these additional components of the composition and suitable agents for pharmaceutical preparation are well known to those skilled in the art. The compositions of the present invention may be prepared using conventional techniques and procedures already well known to the skilled artisan, and for which Ansel's Pharmaceutical Dosage Forms and Drug Delivery Systems, 11^{th} Edition, Wolters Kluwer, Philadelphia (2018), may be used as reference.

In yet a further embodiment, the composition may be in a form suitable for implantation into the eye, conjunctiva, fornix, or eyelid. An implant may also include a microsphere. Such an implant would remain in the tissue for a period of time and release the composition into the surrounding tissue to deliver an effective amount of α adrenergic agonist. Preferably, the period of time is greater than 24 hours and provides for extended or sustained release of the composition. Examples of extended release forms may include vesicles or liposomes, other polymer formulations, and microparticles. The method for implant preparation is well known to those skilled in the art. An example of a delivery device for said implant has been previously described in U.S. Pat. No. 6,899,717 and is incorporated by reference. U.S. Pat. No. 9,504,653 discloses novel methods for making microspheres suitable for ocular implantation, and is incorporated herein by reference.

Another aspect of the invention provides kits for carrying out any of the aforementioned methods. The kits provided herein are also directed to the storage and packaging of the composition. In several embodiments, the kits comprise a container that holds the composition, a container that holds the composition and serves as a delivery device, and a delivery device for administration to the eye, eyelid, or fornix. The delivery device for administration is also contained in a container or package. The delivery device of the kit will depend on whether the composition is administered topically or locally. For illustrative purposes only, a kit may include a multi-use container that is an eyedrop bottle of volume 5 mL, which contains the composition as an aqueous solution and allows for administration to the fornix of the affected side(s). The kits may also include containers that further facilitate storage of the composition. The kits further comprise instructions for use and storage intended for the user and/or health care professional according to the methods disclosed herein. The kits further comprise a list of ingredients, manufacturer, administration, reasons for use or protocols, potential side effects, and toxicity warnings.

While the invention has been described in detail and with reference to several embodiments and certain preferred embodiments, it should be understood that the invention is not limited thereto and that other variations are within the scope of the present invention. Various examples of specific embodiments are presented below and are merely illustrative. It should be appreciated that these specific embodiments do not limit the scope of the invention in any way. Modifications and combinations of the examples below, as well as other embodiments of the invention, will be apparent to one of ordinary skill in the art.

### EXAMPLES

### Example 1

Randomized, Controlled Study of Blepharoptosis Treatment with Naphazoline 0.1%

To evaluate the effects of an ophthalmic solution containing an α adrenergic agonist on blepharoptosis, a randomized, double-blinded, controlled study was conducted using naphazoline 0.1%. Participants with either unilateral or bilateral upper eyelid blepharoptosis were randomized to receive either control eye drops (control group) or an ophthalmic solution containing active ingredient naphazoline 0.1% (treatment group).

The composition of the treatment group included naphazoline hydrochloride (concentration 0.1% w/v), sodium chloride, boric acid, calcium chloride, magnesium chloride, potassium chloride, sodium borate, PURITE®, methylcellulose, and purified water. The ophthalmic drops of the control group were composed of all of the preceding compounds except for naphazoline hydrochloride.

Baseline external photographs of all participants were obtained before eye drop administration. Visual field and contrast sensitivity testing were also tested at baseline. Visual field testing was conducted using an OCULUS Easyfield® perimeter. Contrast sensitivity was tested using a Pelli-Robson Contrast Sensitivity Chart (Pelli D G, Robson J, Wilkins A J. The design of a new letter chart for measuring contrast sensitivity. Clin Vision Sci. 1988; 2187-99). After one eye drop was administered to one or both eyes depending on the presence of unilateral or bilateral blepharoptosis, a second photo was obtained 30 minutes later. After 60 minutes, visual field and contrast sensitivity tests were repeated. After 120 minutes, a third and final photo was obtained.

Blepharoptosis in all participants was functional, defined as a marginal reflex distance-1 (MRD-1) of 2 mm or less. MRD-1 is the distance in millimeters from the upper eyelid margin to the light reflex within the pupil. Measurements were made using Image J software (US National Institutes of Health, Bethesda, MD, U.S.A., http://imagej.nih.gov/ij) by a single designated, blinded investigator. The pupil diameter was also measured at all time points. Secondary effects were recorded and included subject-reported eye irritation, pain, blurry vision, light sensitivity, as well as examiner-noted eye injection, eyelid retraction, and pupil diameter. MRD-1 at the two post-administration time points constituted the primary endpoint. Secondary endpoints included visual field assessment and contrast sensitivity testing.

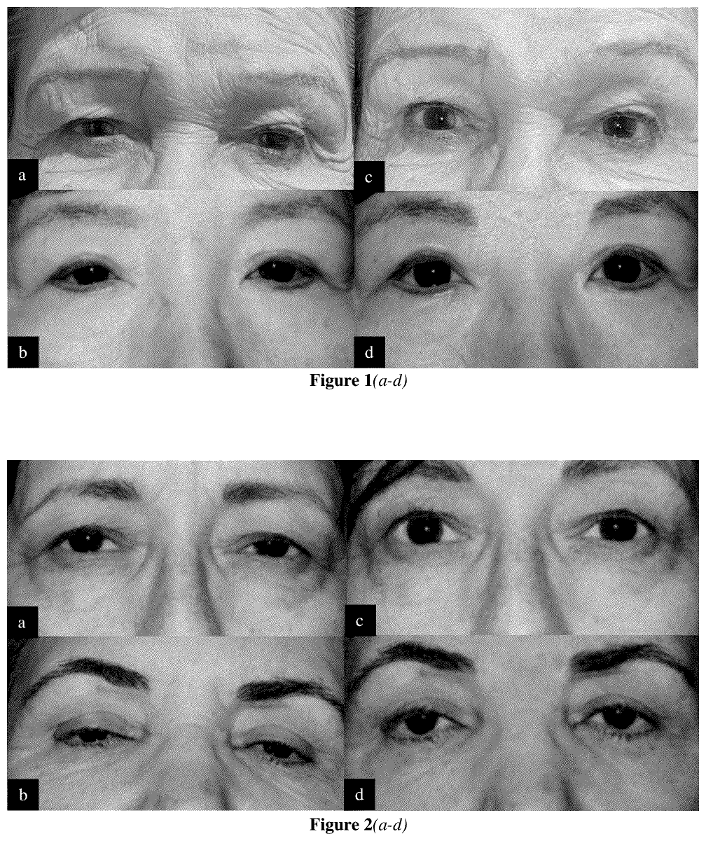

Results are presented in Table 1. In total there were 21 subjects in the control group and 19 subjects in the treatment group. Individuals in the treatment group showed a statistically significant improvement in MRD-1 at 30 minutes and 120 minutes compared to the control group. FIG. 1(a-d) shows external photographs of two subjects with functional blepharoptosis in both eyes before (FIG. 1a-b) and 30 minutes after drop administration of an ophthalmic solution containing naphazoline 0.1% to both eyes (FIG. 1c-d). FIG. 2(a-d) shows external photographs of two other subjects with functional blepharoptosis in both eyes before (FIG. 2a-b) and 90 minutes after drop administration of naphazoline 0.1% ophthalmic solution to both eyes (FIG. 2c-d), demonstrating blepharoptosis in all eyes of subjects in the treatment group.

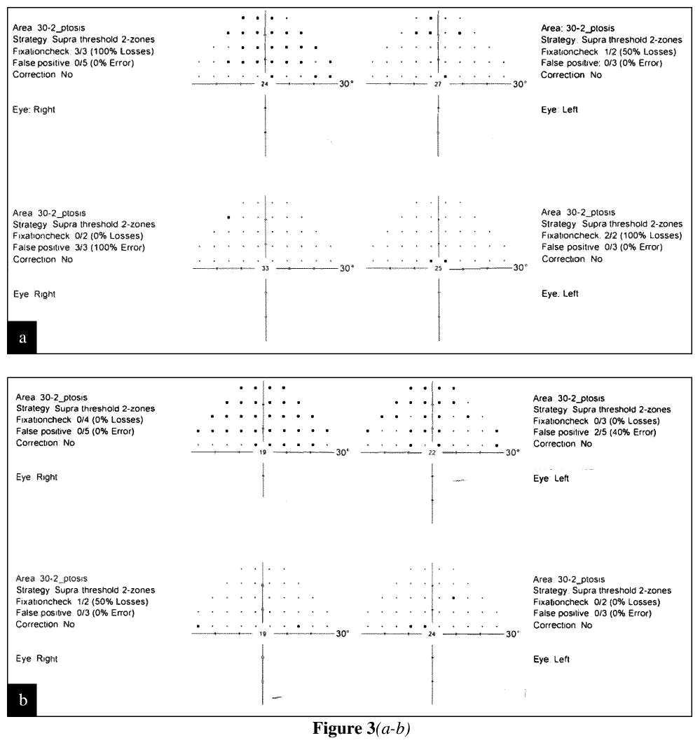

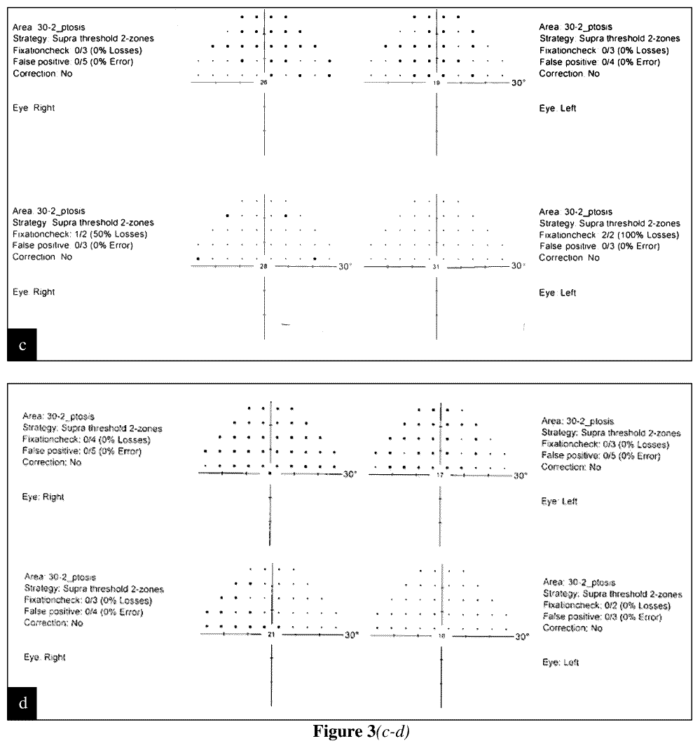

Visual field and contrast sensitivity improvement were greater in the group that received the ophthalmic solution containing naphazoline 0.1% compared to the control group. FIG. 3(a-d) presents superior visual field results in four subjects with functional blepharoptosis. The solid square represents a tested visual field point that was not seen by a subject, while a dot represents a point that was seen by a subject. The top panel of each test result shows visual field results in each eye at baseline, while the bottom panel shows results 60 minutes after drop administration of naphazoline 0.1% ophthalmic solution to both eyes.

Though there was a greater proportion of individuals who reported minor side effects in the treatment group than in the control group, this difference was not statistically significant (Table 1). The study not only demonstrates the superior effects of naphazoline 0.1% ophthalmic solution compared to the control on blepharoptosis, visual field, and contrast sensitivity, but also the longevity of effect.

TABLE 1
Functional Blepharoptosis Outcomes in Control vs. Treatment with Naphazoline 0.1%

| | Control Group | Treatment Group | P value |
|---|---|---|---|
| Subject Age (mean, years) | 54.8 | 56.1 | 0.32 |
| Baseline MRD1 (mean, mm) | 0.97 | 0.93 | 0.41 |
| MRD1 Δ 30 minutes (mean, mm) | 0.02 | 2.15 | <0.001* |
| MRD1 Δ 120 minutes (mean, mm) | 0.01 | 2.08 | 0.009* |
| Baseline Visual Field (VF) (mean, % correct) | 35.2 | 33.8 | 0.09 |
| VF Δ (mean, % correct) | 2.1 | 56.6 | <0.001* |
| Baseline Contrast Sensitivity (mean) | 1.32 | 1.29 | 0.22 |
| Contrast Sensitivity Δ (mean) | 0.03 | 0.69 | 0.03* |
| Secondary Effects (%) | 9.5 | 10.5 | 0.17 |

*statistically significant (p < 0.05)

### Example 2

Randomized, Controlled Study of Blepharoptosis Treatment with Tetrahydrozoline 0.1%

To evaluate the effects of an ophthalmic solution containing an α adrenergic agonist (tetrahydrozoline) on blepharoptosis, a randomized, controlled study was conducted. Participants with both unilateral or bilateral blepharoptosis were randomized to receive either the control eye drop or an ophthalmic solution containing active ingredient tetrahydrozoline 0.1%. Subjects and drop administrators were blinded to the intervention. Blepharoptosis in all participants was functional, defined as a marginal reflex distance-1 (MRD-1) of 2 mm or less.

The composition of the treatment group included tetrahydrozoline hydrochloride (concentration 0.1% w/v), sodium chloride, boric acid, calcium chloride, magnesium chloride, potassium chloride, sodium borate, PURITE®, methylcellulose, and purified water. The ophthalmic drops of the control group were composed of all of the preceding compounds except for tetrahydrozoline hydrochloride.

Baseline external photographs of all participants were obtained before eye drop administration. After one eye drop was administered, a second photo was obtained 30 minutes later. After 90 minutes, a third and final photo was obtained. The pupil diameter in millimeters was also measured in photos at all time points. Measurements were made using Image J software by a single designated, blinded investigator. Secondary effects from the intervention were recorded including subject-reported eye irritation, pain, blurry vision, light sensitivity, as well as examiner-noted eye injection, eyelid retraction, and pupil size change from baseline.

The results of the study demonstrated improved blepharoptosis in the treatment group with tetrahydrozoline 0.1% compared to the control group (Table 2). There were 10 subjects in the control group and 11 in the treatment group. Blepharoptosis improvement was statistically significant at both time points, demonstrating the sustained effect of the α adrenergic agonist on blepharoptosis improvement. Although there was a slighter higher proportion of individuals who reported minor side effects in the treatment group than the control group, this difference was not statistically significant. Pupillary size change was also greater in the treatment group compared to the control, however, this change was not statistically significant.

TABLE 2
Functional Blepharoptosis Outcomes in Control
vs. Treatment with Tetrahydrozoline 0.1%

| | Control Group | Treatment Group | P value |
|---|---|---|---|
| Subject Age (mean, years) | 61.0 | 63.8 | 0.54 |
| Mean Baseline MRD1 (mean, mm) | 0.89 | 0.85 | 0.12 |
| MRD1 Δ 30 minutes (mean, mm) | 0.04 | 2.01 | 0.01* |
| MRD1 Δ 120 minutes (mean, mm) | 0.02 | 1.97 | 0.009* |
| Pupil Size Δ 30 minutes (mean, %) | 5.8 | 9.3 | 0.09 |
| Minor Side Effects (%) | 2.1 | 6.7 | 0.07 |

*statistically significant (p < 0.05)

### Example 3

Case Study of Blepharoptosis Treatment with Brimonidine 0.2%

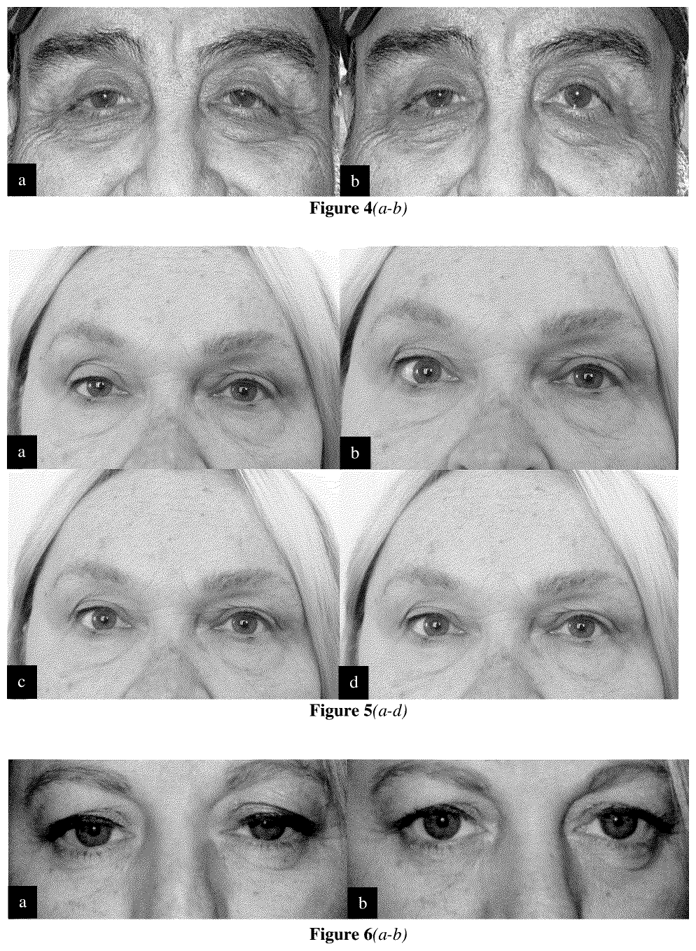

A 70-year-old male complained of left eyelid droopiness and decreased vision of the left eye. On exam, the subject had significant blepharoptosis of the left eye compared to the right eye, with a marginal reflex distance-1 (MRD-1) of 1.5 mm in the right eye and 0.5 mm in the left eye (FIG. 4a). On further testing, the superior visual field of the left eye was limited and contrast sensitivity testing was decreased. The subject was unable to undergo surgical repair due to an underlying heart condition. He was given ophthalmic drops, Alphagan® containing 0.2 percent brimonidine, which was administered twice daily to the left eye. At one-month follow-up, the subject's MRD-1 of the left eye was 3.5 mm (FIG. 4b). On repeat testing, his superior visual field results and contrast sensitivity improved. He also did not endorse any secondary effects.

### Example 4

Case Study of Cosmetic Blepharoptosis Treatment with Naphazoline 0.1%

A 55-year-old subject presented with cosmetic blepharoptosis of the right eye with a marginal reflex distance-1 (MRD-1) of 2.5 mm in the affected eye (FIG. 5a). She also had brow asymmetry due to compensatory right eyebrow elevation, a separate cosmetic concern. One drop of an ophthalmic solution containing naphazoline 0.1% was administered to the right eye. After 30 minutes, the eyelid was over-elevated with an MRD-1 of 5.5 mm due to continued compensatory brow elevation of the subject's right eyebrow (FIG. 5b). After brow relaxation, the subject achieved both eyelid and brow symmetry, with an MRD-1 of the right eye equal to that of the left (FIG. 5c). Three hours after drop administration, the eyelid and brow symmetry were maintained (FIG. 5d).

### Example 5

### Controlled Study of Topical Naphazoline 0.1% to Improve Cosmetic Blepharoptosis

A comparative study was conducted to investigate the effect of an ophthalmic solution containing naphazoline 0.1% on cosmetic blepharoptosis. Two groups, the control group and treatment group each consisting of 5 subjects, were matched in terms of age and gender. The control group received one drop of normal saline while the treatment group received one drop of an ophthalmic solution containing naphazoline 0.1%. At both 30 minutes and 120 minutes after drop administration, external photographs were obtained and analyzed using Image J imaging software. MRD-1 and the distance between the upper and lower eyelid margins were measured to quantitatively assess cosmetic blepharoptosis. Secondary effects were also recorded, including conjunctival injection, eye pain or irritation, light sensitivity, and pupil size.

Results presented in Table 3 show that the treatment group experienced a statistically significant improvement in MRD-1 and eyelid separation compared to the control group as both 30 and 120 minutes. FIG. 6 shows an external photograph of a subject in the treatment group before (FIGS. 6a) and 30 minutes after drop administration (FIG. 6b). The subject demonstrated both an increase in MRD-1 as well as eyelid margin separation, indicative of improvement in cosmetic blepharoptosis and eyelid appearance. There was no difference in secondary effects, including pupil size, between the two groups.

TABLE 3
Cosmetic Blepharoptosis Outcomes in Control vs. Treatment Groups

| | Control Group | Treatment Group | P value |
|---|---|---|---|
| Subject Age (mean, years) | 46.9 | 48.1 | 0.93 |
| Baseline MRD1 (mean, mm) | 2.6 | 2.4 | 0.86 |
| MRD1 Δ 30 minutes (mean, %) | 3.8 | 42 | 0.01* |
| MRD1 Δ 120 minutes (mean, %) | 1.9 | 38 | 0.02* |
| Eyelid Margin Distance Δ 30 minutes | 0.03 | 1.45 | 0.009* |
| (mean, mm) | | | |
| Eyelid Margin Distance Δ 120 minutes | 0.02 | 1.33 | 0.03* |
| (mean, mm) | | | |
| Pupil Size Δ 30 minutes (mean, %) | 6.7 | 10 | 0.37 |
| Minor Side Effects (%) | 20 | 20 | 0.49 |

*statistically significant (p < 0.05)

What is claimed:

1. A method for treating blepharoptosis in an individual in need thereof comprising administering a therapeutically effective amount of an α adrenergic receptor agonist or a pharmaceutically acceptable salt thereof.

2. The method of claim 1, wherein the α adrenergic receptor agonist is administered topically or locally to the eye, eyelid, fornix, or any combination thereof.

3. The method of claim 1, wherein the α adrenergic receptor agonist is selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof.

4. The method of claim 1, wherein the α adrenergic receptor agonist is selected from naphazoline, tetrahydrozoline, phenylephrine, amidephrine, cirazoline, desglymidodrine, metaraminol, metoxamine, midodrine, xylometazoline, α-methyldopa, apraclonidine, brimonidine, clonidine, dexmedetomidine, guanabenz, guanfacine, medetomidine, xylazine, α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, dipivefrin, epinephrine, isoproterenol, mephentermine, norepinephrine, pseudoephedrine, or any combination thereof.

5. The method of claim 3, wherein the selective α₁ adrenergic receptor agonist is selected from naphazoline, tetrahydrozoline, phenylephrine, amidephrine, cirazoline, desglymidodrine, metaraminol, metoxamine, midodrine, xylometazoline, or any combination thereof.

6. The method of claim 3, wherein the selective α₂ adrenergic receptor agonist is selected from α-methyldopa, apraclonidine, brimonidine, clonidine, dexmedetomidine, guanabenz, guanfacine, medetomidine, xylazine, or any combination thereof.

7. The method of claim 3, wherein the agonist with α₂ adrenergic receptor activity is selected from α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, or any combination thereof.

8. The method of claim 3, wherein the non-selective α₁/α₂ adrenergic receptor agonist is selected from dipivefrin, epinephrine, isoproterenol, mephentermine, norepinephrine, pseudoephedrine, or any combination thereof.

9. The method of claim 1, wherein the α adrenergic receptor agonist is administered in a pharmacologically acceptable form selected from a solution, dispersion, suspension, emulsion, microemulsions, ointment, gel, hydrogel, jelly, liposome, nanoparticle, lotion, cream, paste, spray, foam, microsphere, or solid implant.

10. The method of claim 1, wherein the α adrenergic receptor agonist is administered in a pharmaceutically acceptable carrier as a composition with at least one other ingredient selected from lubricating or thickening agents, buffering agents, pH adjustors, tonicity adjustors, stabilizers, antioxidants, preservatives, sequestering agents, components included in artificial tears, or any combination thereof.

11. A method for improving the cosmetic appearance of the eyes, eyelids, eyebrows, or any combination thereof in an individual in need thereof comprising administering a therapeutically effective amount of an α adrenergic receptor agonist or a pharmaceutically acceptable salt thereof.

12. The method of claim 11, wherein the α adrenergic receptor agonist is administered topically or locally to the eye, eyelid, fornix, or any combination thereof.

13. The method of claim 11, wherein the α adrenergic receptor agonist is selected from a selective α₁ adrenergic receptor agonist, a selective α₂ adrenergic receptor agonist, an agonist with α₂ adrenergic receptor activity, a non-selective α₁/α₂ adrenergic receptor agonist, or any combination thereof.

14. The method of claim 11, wherein the α adrenergic receptor agonist is selected from naphazoline, tetrahydrozoline, phenylephrine, amidephrine, cirazoline, desglymidodrine, metaraminol, metoxamine, midodrine, xylometazoline, α-methyldopa, apraclonidine, brimonidine, clonidine, dexmedetomidine, guanabenz, guanfacine, medetomidine, xylazine, α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, dipivefrin, epinephrine, isoproterenol, mephentermine, norepinephrine, pseudoephedrine, or any combination thereof.

15. The method of claim 13, wherein the selective α₁ adrenergic receptor agonist is selected from naphazoline, tetrahydrozoline, phenylephrine, amidephrine, cirazoline, desglymidodrine, metaraminol, metoxamine, midodrine, xylometazoline, or any combination thereof.

16. The method of claim 13, wherein the selective α₂ adrenergic receptor agonist is selected from α-methyldopa, apraclonidine, brimonidine, clonidine, dexmedetomidine, guanabenz, guanfacine, medetomidine, xylazine, or any combination thereof.

17. The method of claim 13, wherein the agonist with α₂ adrenergic receptor activity is selected from α-methylnorepinephrine, amphetamine, dextroamphetamine, ethylnorepinephrine, ephedrine, epinine, levarterenol, lofexidine, methamphetamine, methylphenidate, mivazerol, moxonidine, norepinephrine, norphenylephrine, pemoline, phenylpropanolamine, propylhexedrine, tizanidine, or any combination thereof.

18. The method of claim 13, wherein the non-selective α₁/α₂ adrenergic receptor agonist is selected from dipivefrin, epinephrine, isoproterenol, mephentermine, norepinephrine, pseudoephedrine, or any combination thereof.

19. The method of claim 11, wherein the α adrenergic receptor agonist is administered in a pharmacologically acceptable form selected from a solution, dispersion, suspension, emulsion, microemulsions, ointment, gel, hydrogel, jelly, liposome, nanoparticle, lotion, cream, paste, spray, foam, microsphere, or solid implant.

20. The method of claim 11, wherein the α-adrenergic receptor agonist is administered in a pharmaceutically acceptable carrier as a composition with at least one other ingredient selected from lubricating or thickening agents, buffering agents, pH adjustors, tonicity adjustors, stabilizers, antioxidants, preservatives, sequestering agents, components included in artificial tears, or any combination thereof.
